(19) United States
(12) Patent Application Publication
(10) Pub. No.: US 2017/0209701 A1
(43) Pub. Date: Jul. 27, 2017

(54) SYSTEMS AND METHODS FOR CONTROLLING A VENTRICULAR RATE DURING ATRIAL FIBRILLATION

(71) Applicant: INDIANA UNIVERSITY RESEARCH & TECHNOLOGY CORPORATION, Indianapolis, IN (US)

(72) Inventor: Peng-Sheng Chen, Indianapolis, IN (US)

(21) Appl. No.: 15/328,863

(22) PCT Filed: Jul. 24, 2015
(86) PCT No.: PCT/US2015/041979

§ 371 Date: Jan. 24, 2017

Related U.S. Application Data
(60) Provisional application No. 62/029,116, filed on Jul. 25, 2014.

(51) Int. Cl. A61N 1/36

(52) U.S. Cl. CPC A61N 1/36114; A61N 1/36146; A61N 1/36135

(57) ABSTRACT

Systems and methods for controlling a ventricular rate during atrial fibrillation are provided. In some aspects, a method includes selecting a treatment protocol based on a determined condition of a subject, the treatment protocol comprising intermittent periods of electrical stimulation separated by periods of non-stimulation. The method also includes applying an electrical stimulation, according to the selected the treatment protocol, using an electrical source and at least one electrode positioned at locations associated with a vagal nerve of the subject to control a ventricular rate during atrial fibrillation.

### GOVERNMENT RIGHTS

This application is based on, claims priority to, and incorporates herein by reference in its entirety U.S. Provisional Application Ser. No. 62/029,116, filed Jul. 25, 2014, and entitled “SYSTEMS AND METHODS FOR CONTROLLING A VENTRICULAR RATE DURING ATRIAL FIBRILLATION.”

### GOVERNMENT RIGHTS

This invention was made with government support under HL071140 awarded by National Institutes of Health. The government has certain rights in the invention.

### BACKGROUND

The present disclosure relates generally to systems and methods for electrical stimulation of nerves, and, more specifically, to systems and methods for electrically stimulating a vagus nerve to control a heart function.

Atrial fibrillation (“AF”), either paroxysmal or persistent, is one of the most common heart rhythm affliction faced by patients in the United States. The symptoms of AF vary considerably, ranging from minimal or no symptoms, to incapacitating palpitations or congestive heart failure. Although the factors responsible for the severity of symptoms in AF are poorly delineated, they include excessive tachycardia, the presence and severity of underlying heart disease, and individual variations in patient awareness of the irregular rhythm. Specifically, one of the main consequences of AF includes a rapid ventricular response resulting in an increased ventricular rate. Since the main pumping function in the heart is performed by the ventricles, an excessive ventricular rate can have serious side effects on cardiac performance and hence patient outcomes.

Some management strategies for AF have included pharmacological approaches, either targeting the prevention of AF altogether, known as rhythm control, or reducing the ventricular rate, a method known as rate control. In particular, ventricular rate involves a simpler strategy with fewer medical procedures and less toxic medications. Nevertheless, clinical trials have shown that both rate control and rhythm control are equally effective in managing patients with AF.

In some patients, however, neither rhythm control nor rate control have been shown to be effective using pharmacological methods. Hence, alternative approaches have been developed for treatment of AF, including chronic electrical stimulation using a selected current, voltage, frequency, and waveform. In humans and various vertebrates, the autonomic nervous system includes a sympathetic nervous system and a parasympathetic nervous system. Together, the sympathetic and parasympathetic nervous systems are understood to regulate many bodily activities including heart rhythm, skeletal muscle contraction, and bowel activity. Since the heart is innervated by a parasympathetic nervous system through the cardiac branch of the vagus nerve, treatment methods have targeted the vagus nerve, using electrical stimulation to produce a particular response. Such vagal nerve stimulation (“VNS”) methods have been utilized to prevent ventricular fibrillation and sudden cardiac death, as well as improve cardiac autonomic control and significantly attenuate heart failure symptoms in humans and some mammals.

A slower ventricular rate, referring specifically to ventricular contractions, during sinus rhythm and during AF may be helpful in managing patients with heart failure or atrial arrhythmias. However, the manner in which VNS affects changes in ventricular rate during AF, as well as the incidence of atrial arrhythmias, ventricular arrhythmias and cardiac contractile performance, are incompletely understood. One proposed mechanism stipulates that VNS activates the parasympathetic nerves within the vagal nerve, which releases acetylcholine, a biochemical believed to be directly responsible for reducing the heart rate. Assuming such mechanism, continuous VNS or closed-loop VNS methods have been employed to achieve control of ventricular rate during AF. However, such approaches are associated with significant complications due to continuous electrical stimulation of the vagal nerve, and additional complications and cost due to hardware and software required to sense ventricular rate and deliver stimulus when the rate falls below certain threshold.

Therefore, given the above, there remains a need for systems and methods for efficiently controlling a ventricular rate during atrial fibrillation.

### SUMMARY

The present invention overcomes the aforementioned drawbacks by providing systems and methods directed to monitoring and controlling a medical condition of a subject. In particular, a novel approach is described whereby a treatment protocol involving intermittent periods of electrical stimulation can be applied to a subject to control the subject's condition. For instance, in some aspects, the treatment protocol may include intermittent vagal nerve stimulation (“VNS”) configured to reduce stellate ganglion nerve activity (“SGNA”) to control ventricular rate of the subject during atrial fibrillation (“AF”).

As will be appreciated from the following descriptions, the approach presented herein avoids complications associated with providing continuous electrical stimulation to the vagal nerve. In addition, it allows for an open-loop operation of VNS, providing a simplified clinical application for controlling a ventricular rate during AF without need for implantation of additional electrodes or devices to sense the heart rate in the ventricles.

In accordance with one aspect of the disclosure, a system for controlling a ventricular rate during atrial fibrillation is provided. The system includes one or more electrodes positioned at locations associated with a vagal nerve of the subject, and an electrical source configured to electrically stimulate the vagal nerve to control a ventricular rate during atrial fibrillation. The system also includes a processor configured to select a treatment protocol based on a determined condition of the subject, wherein the treatment protocol comprises intermittent periods of electrical stimulation separated by periods of non-stimulation, and apply an electrical stimulation, according to the selected the treatment protocol, using the electrical source and the one or more electrodes.

In accordance with another aspect of the disclosure, a method for controlling a ventricular rate during atrial fibrillation is provided. The method includes selecting a treatment protocol based on a determined condition of a subject, the treatment protocol comprising intermittent periods of electrical stimulation separated by periods of non-stimulation. The method also includes applying an electrical stimulation, according to the selected the treatment protocol, using an electrical source and at least one electrode positioned at locations associated with a vagal nerve of the subject to control a ventricular rate during atrial fibrillation

The foregoing and other advantages of the invention will appear from the following description.

### BRIEF DESCRIPTION OF THE DRAWINGS

FIG. 1 is an anatomical diagram showing the anatomically accepted structure of the vague nerve extending from the cranial cavity to the abdominal cavity in a human.

FIG. 2 is an anatomical diagram showing the anatomically accepted structure of the vague nerve as it relates to a typical human heart.

FIG. 3 is a block diagram depicting a process of vagal nerve stimulation, in accordance with aspect of the present disclosure.

FIG. 4 is a system for vagal nerve stimulation in accordance with aspects of the present disclosure.

FIG. 5A is a schematic illustrating an example treatment protocol in accordance with aspects of the present disclosure.

FIG. 5B is a schematic illustrating another example treatment protocol in accordance with aspects of the present disclosure.

FIG. 6A is a graphical illustration depicting example neural activity and ventricular rate modifications as a result of treatment in accordance with aspects of the present disclosure.

FIG. 6B is a graphical illustration depicting another example neural activity and ventricular rate modifications as a result of treatment in accordance with aspects of the present disclosure.

FIG. 7A is a graphical illustration depicting neural activity and ventricular rate at baseline sinus rhythm.

FIG. 7B is a graphical illustration depicting neural activity and ventricular rate during atrial fibrillation.

FIG. 7C is a graphical illustration depicting an example of neural activity and ventricular rate modifications as a result of treatment in accordance with aspects of the present disclosure.

FIG. 7D is a graphical illustration depicting another example of neural activity and ventricular rate modifications as a result of treatment in accordance with aspects of the present disclosure.

FIG. 8A is a graphical illustration comparing stellate ganglion nerve activity at baseline, during atrial fibrillation, and a result of treatment in accordance with aspects of the present disclosure.

FIG. 8B is a graphical illustration comparing vagal nerve activity at baseline, during atrial fibrillation, and a result of treatment in accordance with aspects of the present disclosure.

FIG. 8C is a graphical illustration comparing heart rate at baseline, during atrial fibrillation, and as a result of treatment in accordance with aspects of the present disclosure.

FIG. 9A is a graphical illustration depicting an example of prolonged pauses in nerve activity during atrial fibrillation.

FIG. 9B is a graphical illustration depicting another example of prolonged pauses in nerve activity during atrial fibrillation.

FIG. 10A shows a low magnification image of an example left ganglion histological sample stained with tyrosine hydroxilase (“TH”) immunostaining.

FIG. 10B shows a low magnification image of an example left ganglion histological sample stained using trichrome staining.

FIG. 10C shows a high magnification image of the sample of FIG. 10A, indicating stellate ganglion remodeling from treatment in accordance with aspects of the present disclosure.

FIG. 10D shows a high magnification image of the sample of FIG. 10B, indicating stellate ganglion remodeling from treatment in accordance with aspects of the present disclosure.

FIG. 11A shows an image of an example left stellate ganglion histological TH-stained sample from a normal dog

FIG. 11B shows an image of an example left stellate ganglion histological TH-stained sample from a dog subjected to pacing-induced atrial fibrillation.

FIG. 11C shows an image of an example right stellate ganglion histological TH-stained sample from a dog subjected to pacing-induced atrial fibrillation.

FIG. 11D shows an image of an example left stellate ganglion histological TH-stained sample from a dog subjected to treatment, in accordance with aspects of the present disclosure.

FIG. 11E is a graphical illustration comparing the percentage of TH-negative cells in each animal group.

FIG. 12 shows confocal images for the sample of FIG. 10A stained with TH staining and terminal deoxynucleotidyl transferase dUTP nick end labeling (“TUNEL”) staining, comparing normal and damaged regions.

FIG. 13 is a graphical illustration comparing RR interval distribution curves for the different animal groups studied.

### DETAILED DESCRIPTION

Controlling a ventricular rate during sustained atrial fibrillation (“AF”) has been found to be difficult in some patients via traditional pharmacological treatments. Hence, alternative approaches have resorted to cervical vagal nerve stimulation (“VNS”) to control the ventricular rate, with the idea being that VNS activates the parasympathetic nervous system, thus reducing the ventricular rate, for instance, during AF. To ensure that the ventricular rate is reduced, these approaches typically utilize either a continuous VNS or “closed loop” electrical stimulation of the vagal nerve in order to achieve the therapeutic goals. Specifically, a closed loop approach requires sensing wires to be inserted into the ventricles of a subject's heart in order to provide feedback for determining the ventricular rate. When the measured rate exceeds a certain threshold, a VNS treatment is activated to suppress the ventricular rate. As described, continuous VNS is associated with significant side effects, while closed-loops VNS requires additional hardware and software to sense ventricular rate and deliver the stimulus. Also, such treatment approach relies on the assumption that VNS activates the parasympathetic nerves within the vagal nerve, which then releases the biochemical acetylcholine to reduce the heart rate.

The present invention recognizes that the vagal nerve includes both sympathetic and parasympathetic components, and hence VNS does not selectively activate only the parasympathetic nerves in the vagus nerve. In addition, VNS can oppose sympathetic actions at both pre and post-junctional levels. Therefore, the present disclosure recognizes that acute electrical stimulation treatment may be provided to inhibit sympathetic nerve firing such that a ventricular rate may be controlled for patients with AF. Also, it is a discovery of the present disclosure that chronic treatment may be provided to remodel the structure and function of one or more neural structures in order to achieve ventricular rate control. For instance, in some implementations, the stellate ganglion may be remodeled using a treatment protocol that modifies or reduces the stellate ganglion nerve activity (“SGNA”), which is believed to be responsible for the accelerated ventricular rate during AF.

In addition, since controlling ventricular rate involves reduction of sympathetic output due to stellate ganglion remodeling, the present invention recognizes that it is then possible to utilize non-continuous VNS to achieve therapeutic effects. In particular, an intermittent VNS process may be applied to control a ventricular rate during AF. Specifically, such process may consist of a number of “ON” and “OFF” treatment periods, referring to time periods during which VNS is active or inactive, respectively. These intermittent “ON” and “OFF” periods may be unequal in duration and, in this regard, the process may be referred to as asynchronous. In some aspects, short ON time periods may be used to remodel the sympathetic nerve structures and hence reduce sympathetic outflow, while long OFF time periods would prevent the side effects present in continuous VNS applications.

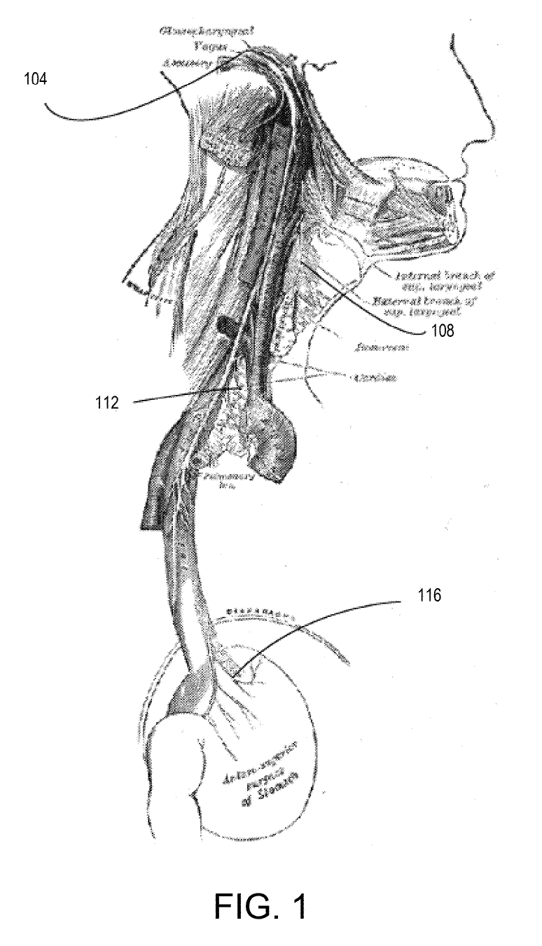

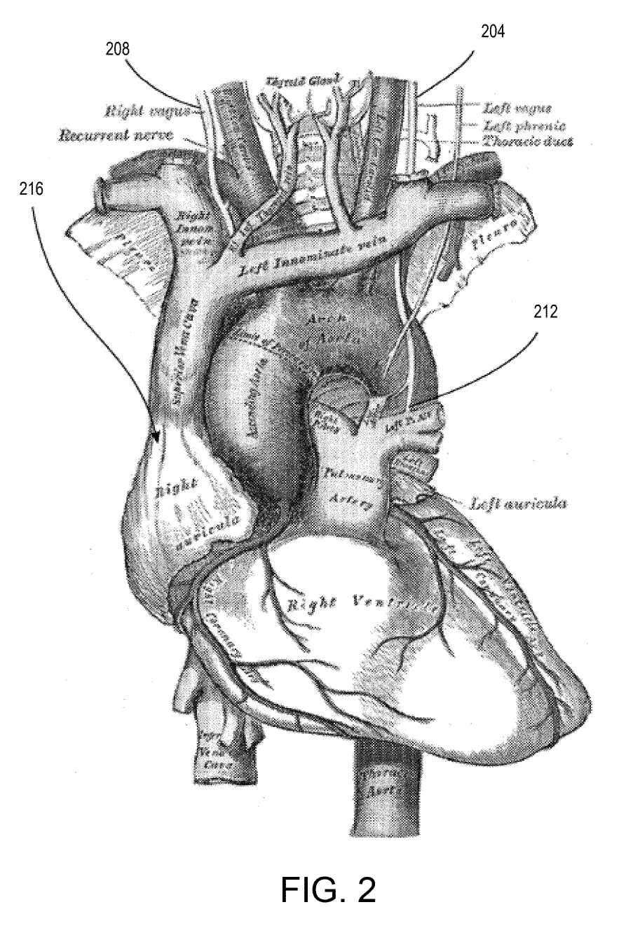

Referring to FIGS. 1 and 2, a simplified depiction of some of the organs, muscles, and systems that interact with the vagus nerve for a human subject is shown. The vagus nerve begins in the cranial cavity as seen in FIG. 1 at 104, and extends throughout much of the thoracic and abdominal cavities, including laryngeal muscles 108, cardiac muscles 112, and the stomach 116. The vagus nerve includes various branches with two major branches being the left vagus and right vagus nerve. As depicted in FIG. 2, the left vagus 204 stimulates the atrioventricular (“AV”) node 212 in the heart, while the right vagus 208 stimulates the sinoatrial node 216. However, the left vagus nerve stimulation can also affect the sinus node 216 while the right vagus nerve stimulation may also affect the AV node 212. Various nerves including the vagus nerves are formed from both afferent and efferent nerve fibers. The afferent nerve fibers conduct nervous signals from tissue or organs in the body to the central nervous system. The efferent nerve fibers conduct nervous signals from the central nervous system to organs or tissue throughout the body.

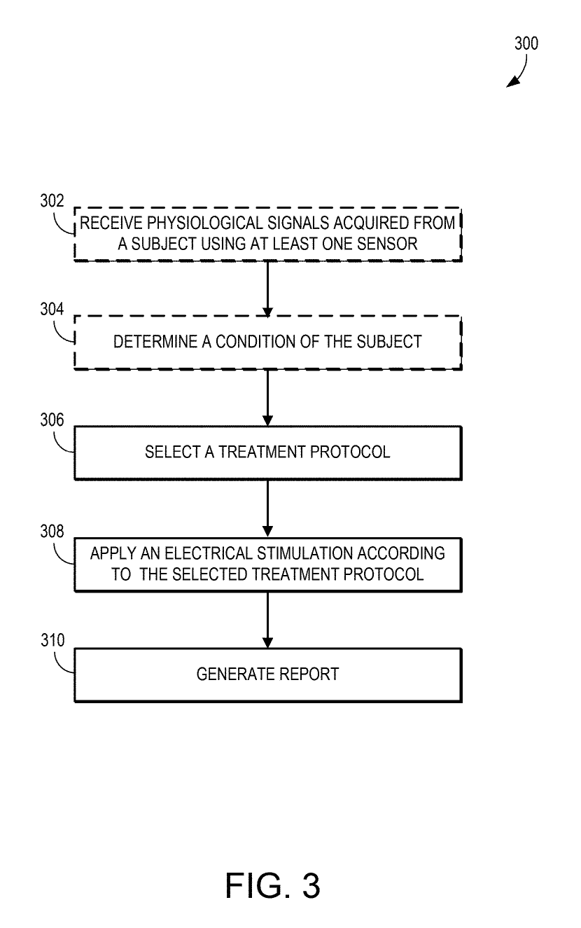

Referring to FIG. 3, the steps of a non-limiting example of a process 300 for controlling a ventricular rate of a subject during an atrial fibrillation, in accordance with the present disclosure, are shown. The process 300 may optionally begin at process block 302 with receiving physiological signals acquired from a subject using at least one sensor. In some aspects, physiological signals may also be acquired at process block 302, and processed as desired. Example physiological signals include signals associated with heart activity, nerve activity, respiratory activity, and so forth. Then, at process block 304, a condition of the subject may be determined. In particular, such determination may be performed by a clinician or by a processor configured to do so, or a combination of both, using physiological data and/or other information associated with the subject, such as one or more patient characteristic, medical history, and so forth. For example, the determined condition can include a cardiac condition, such as atrial fibrillation, a condition associated with a specific nerve activity, and so forth.

At process block 306, a treatment protocol may then be identified or selected based on the determined condition. In particular, the treatment protocol may be configured to include intermittent periods of electrical stimulation separated by periods of non-stimulation. The periods of electrical stimulation may be described by various combinations of parameters, including timing, duration, intensity, frequency, waveform, and combinations thereof. As a non-limiting example, an applied electrical stimulation may have an intensity in a range between 0.5 mA to 5 mA, a frequency between 0.1 Hz and 20 Hz, and a pulse width between 0.1 ms and 5 ms. In some aspects, the periods of electrical stimulation may be longer than the periods of non-stimulation, while in other aspects, the non-stimulation periods may be longer. For instance, time durations of the periods of electrical stimulations may be in a range between 1 and 20 seconds, while the time durations of the periods of non-stimulation may be in a range between 60 seconds to 15 minutes, although other values may be possible.

In accordance with the disclosure, the treatment protocol may be selected at process block 306 to control a ventricular rate of a subject during atrial fibrillation. In some aspects, the treatment protocol may be configured to control a sympathetic nerve activity. For instance, parameters of the electrical stimulation may be selected to reduce a neural activity, such as a stellate ganglion activity. Also, the treatment protocol may be configured to remodel at least one neural structure, such as the stellate ganglion. Then, at process block 308, the electrical stimulation, according to the selected treatment protocol, may then be applied using electrodes positioned at various locations about the subject, including the vagal nerve of the subject.

In some aspects, a report may be generated at process block 310, which may be of any shape or form, and provide any desirable information. For example, the report may be representative of acquired physiological signals, a determined subject condition, a selection provided by a processor or user, a determined electrical stimulation treatment protocol, and/or progress of an electrical stimulation treatment.

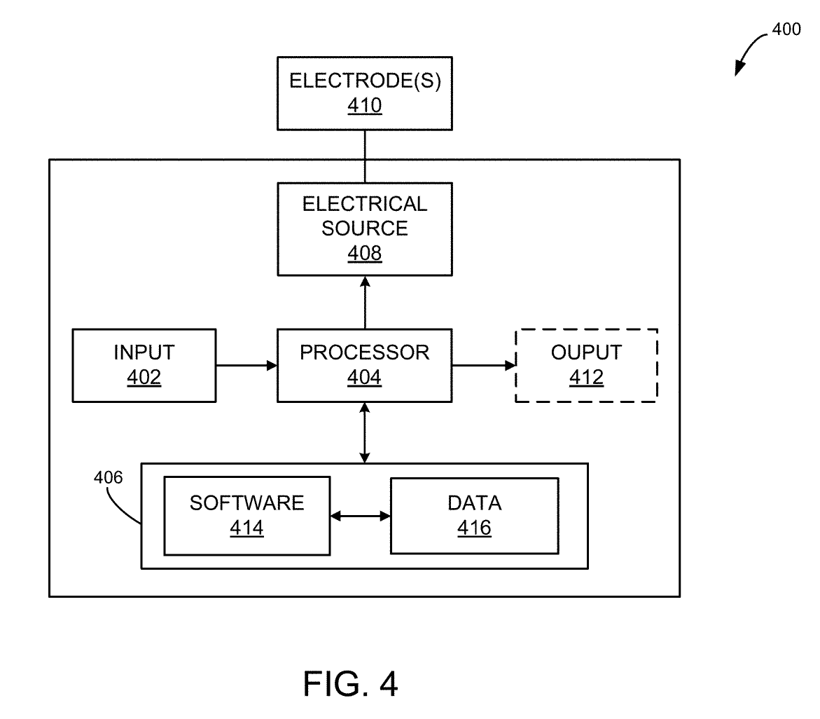

Turning to FIG. 4, a block diagram of an example system 400, for use in accordance with the present disclosure, is shown. In general, the system 400 may be any device, apparatus or system designed with software and hardware capabilities and functionalities for identifying, monitoring and controlling a condition of a subject, such as an elevated ventricular rate during atrial fibrillation. The system 400 may include configurations for operating autonomously, or semi-autonomously according to instructions from a user or clinician, and/or in collaboration with a computer, system, device, machine, mainframe, or server. As shown in FIG. 4, the system 400 may generally include an input 402, at least one processor 404, a memory 406, an electrical source 408 coupled to one or more electrodes 410, and optionally an output 412. In some aspects, the entire system 400, or portions thereof, may be portable, wearable, or implantable.

The input 402 may take any form, and be configured to receive, via wired or wireless connection, a variety of information or data to be processed by the processor 404, including information provided by a user, or information stored on a computer, a server, a database, a hard drive, a CD-ROM, flash memory, or other computer-readable medium. In some embodiments, the input 402 may be configured to receive instructions from a user regarding monitoring or treatment of the subject with the system 400. For example, the input 402 may include capabilities for selecting, entering or otherwise specifying parameters associated with a treatment protocol using electrical stimulation, including timing, duration, intensity, frequency, waveform, and others.

In some configurations, the input 402 may optionally include sensors or electrodes configured to acquire physiological signals from a subject, either intermittently or in real-time. Example physiological signals, include signals associated with nerve activity, cardiac activity, respiratory activity, and so on.

Among capabilities for carrying out processing tasks necessary for operating the system 400, the processor 404 may also be configured to carry out steps to control a subject's condition in accordance with methods described herein. In some embodiments, the processor 404 may be configured to determine the existence or severity of a subject's condition by analyzing physiological information obtained from the subject, and generate a report. In addition, the processor 404 may be configured to direct the acquisition of physiological signals via input 402 or another device or system configured to do so. Example physiological signals include signals associated with nerve activity, cardiac activity, respiratory activity, and so forth. Such physiological information, optionally in combination with input or information provided by a user, may then be analyzed by the processor 404 to determine the existence or severity of a subject's condition. In some aspects, the processor 404 may retrieve physiological information, and other data, from memory 406, or another storage location.

In analyzing the physiological information, the processor 404 may carry out any number of steps for manipulating, filtering, integrating, enhancing, or correcting retrieved or acquired physiological signals. For instance, the processor 404 may be capable of assembling time-series datasets using the physiological signals. In some aspects, the processor 404 may estimate a baseline neural activity, such as a sympathetic nerve activity, or a parasympathetic nerve activity, or a baseline cardiac or respiratory activity, or combinations thereof. For example, the processor 404 may determine an atypical ventricular rate or nerve activity by performing a comparison with one or more baseline values. In some aspects, the processor 404 may adapt an ongoing treatment protocol in accordance with subject progress, which may be identified, for instance, using physiological measurements, as described above.

Based on a subject condition, the processor 404 may be configured to determine or select, either autonomously or semi-autonomously, a treatment protocol involving electrical stimulation. In accordance with the aspects of the disclosure, the treatment protocol can include intermittent periods of electrical stimulation. That is, the treatment protocol can include any number of periods of electrical stimulation, or “ON” periods, as well as a number of non-stimulation, or “OFF” periods. In some aspects, the “ON” and “OFF” periods may be arranged in a temporally periodic fashion, for example, in an alternating fashion, or an non-periodic fashion. Temporal patterns consisting of combinations of periodic and non-periodic “ON” and “OFF” periods may also be possible. In some implementations, the “ON” and “OFF” periods may be unequal in duration, and in this regard, asynchronous. In addition, stimulation parameters associated with each “ON” period need not be identical. For instance, the timing, duration, intensity, frequency, waveform of electrical stimulation delivered may vary from one “ON” period to another. Similarly, “OFF” periods may also vary in duration.

In one non-limiting example, intermittent periods of electrical stimulation may be delivered using electric pulses with frequencies between 0.1 Hz and 20 Hz, pulse widths between 0.1 ms and 5 ms, and stimulation intensities between 0.5 mA to 5 mA, although other values are possible. Additionally, a treatment protocol may include brief ON periods, for example, between 1 to 20 seconds in duration, and long OFF periods, for example, lasting between 60 seconds to 15 minutes in duration, although other values may be possible. In some aspects, stimulation intensities during one or more “ON” periods may vary with time.

In some aspects, a treatment protocol may be assembled by the processor 404 such that a reduced activity of one or more neural structures, including sympathetic structures, can be achieved. In other aspects, a treatment protocol may be configured to induce neural structure remodeling, such as stellate ganglion remodeling. In yet other aspects, the treatment protocol may be customized to the specifics of each subject, for instance, by taking into consideration the determined baseline neural activity, such as a sympathetic nerve activity or parasympathetic nerve activity, a cardiac activity, and a target neural activity or cardiac activity, or ventricular rate.

The memory 406 may contain software 414 and data 416, and may be configured for storage and retrieval of signal data to be processed by the processor 404. In some aspects, the memory 406 may include a number of pre-programmed treatment protocols or regimens. In addition, the software 414 may contain instructions for determining a subject's condition, as well as determining or selecting an electrical stimulation treatment protocol, based on processed physiological information associated with a subject and/or a user selection.

The electrical source 408, in communication with the processor 408, may then receive instructions therefrom to generate and apply an electrical stimulation to a subject, in accordance with the selected or determined treatment protocol, using various electrodes 410 positioned about or coupled to the subject. In some aspects, one or more electrodes 410 are positioned at locations associated with a vagal nerve of the subject. The applied electrical stimulation may then be delivered to the subject therethrough to control a ventricular rate during atrial fibrillation, as described.

As shown in FIG. 4, the system 400 optionally includes an output 412 connected to the processor 404 capable of providing a report of any shape or form. For example, the report may include, in addition to other desired information, information related to received or acquired physiological signals, identified or determined subject condition(s), selection(s) provided by a user, electrical stimulation treatment protocol(s), a progress or status of an electrical stimulation treatment, and so forth.

In accordance with one aspect of the disclosure, a system for controlling a ventricular rate during atrial fibrillation is provided. The system includes one or more electrodes positioned at locations associated with a vagal nerve of the subject, at least one sensor configured acquire physiological signals from the subject, and an electrical source configured to electrically stimulate the vagal nerve to control a ventricular rate during atrial fibrillation. The system also includes a processor configured to receive the physiological signals from the at least one sensor, and determine a condition of the subject using the received physiological signals. The processor is also configured to identify a treatment protocol using the determined condition, the treatment protocol comprising intermittent periods of electrical stimulation separated by periods of non-stimulation, and apply an electrical stimulation, according to the selected the treatment protocol, using the electrical source and the one or more electrodes, to the locations associated with a vagal nerve of a subject.

In accordance with another aspect of the disclosure, a method for controlling a ventricular rate during atrial fibrillation is provided. The method receiving physiological signals acquired from a subject acquired using at least one sensor, and determining a condition of the subject using the received physiological signals. The method also includes identifying a treatment protocol using the determined condition, the treatment protocol comprising intermittent periods of electrical stimulation separated by periods of non-stimulation, and applying an electrical stimulation, using the treatment protocol and at least one electrode, to locations associated with a vagal nerve of a subject to control a ventricular rate during atrial fibrillation.

The above-described system and method may be further understood by way of examples. These examples are offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims. For example, certain arrangements and configurations are presented, although it may be understood that other configurations may be possible, and still considered to be well within the scope of the present invention. Likewise, specific process parameters and methods are recited that may be altered or varied based on variables such as signal amplitude, phase, frequency, duration, and so forth.

### EXAMPLE

A study was performed to validate that intermittent VNS with a brief ON time and a long OFF time is effective in controlling the ventricular rate by reducing the SGNA. Specifically, a left cervical VNS was performed for 9 mongrel dogs while recording the left SGNA and vagal nerve activity (“VNA”). In the first 3 dogs, an intermittent VNS (14 s ON-time, 10 Hz, 0.5 ms pulse width) with stimulation intensity ranging from 0.5 mA to 2.5 mA and OFF-time ranging from 66 s to 10 m was given during sinus rhythm. Results showed that 1.5 mA output, 14-s ON, 66 s and 3 min OFF-time were effective in suppressing SGNA. A sustained AF was then induced in the remaining 6 dogs using continuous rapid (10 Hz) atrial pacing for two weeks, followed by VNS with 14-s ON and 66-s OFF or 3-min OFF. The integrated SGNA (“iSGNA”) and ventricular rate during AF at baseline were 4.8±2.2 mV-s and 142±33 bpm, respectively. VNS reduced iSGNA and ventricular rate during AF to 3.7±1.8 mV-s (p=0.021) and 115±24 bmp (p=0.016) in VNS with 66-s OFF time, and to 4.1±2.0 mV-s (p=0.037) and 114±39 bpm in 3-min OFF-time (p=0.039). Prolonged (>3s) pauses were observed with increased frequency during VNS as compared with baseline. These prolonged pauses were found 89±79 times/24 hours and 213±245 times/24 hours during VNS 66 s-OFF and VNS 3 m-OFF, respectively. In comparison, prolonged pauses were found only 2±2 time/24 hours during baseline sinus rhythm and 16±14 times/24 hours during AF. The iSGNA was lower during the prolonged pause (0.8±0.2 mV-s) than not during the pause (7.4±2.9 mV-s, p=0.06). Tyrosine hydroxylase (“TH”) staining of the left stellate ganglion showed large areas of reduced staining and significantly increased percentage of TH-negative ganglion cells as compared to the right stellate ganglion of the same dogs

### Methods and Materials

### Continuous Ambulatory Autonomic Nerve Recordings

Under isoflurane inhalation general anesthesia, the left cervical vagal nerve was surgically isolated from the carotid artery. A bipolar pacing lead and an anchor were attached around the left cervical vagal nerve and connected to a subcutaneously positioned Cyberonics Demipulse neurostimulator (Cyberonics Inc, Houston, Tex.). Subsequently, a Data Sciences International (DSI; St Paul, Minn.) radiotransmitter D70CCTP (1 Group 1 dog) and D70EEE (all other dogs) were implanted to record nerve activity through the left 4th intercostal space according to methods described in detail elsewhere. All dogs had one pair of bipolar electrodes sutured onto the left stellate ganglion (“LSG”) beneath its fascia to record SGNA and a second pair of bipolar electrodes inserted into the left ventricular free wall to record the intracardiac electrocardiogram. In 8 dogs with D70EEE devices, a third bipolar pair was used to record left vagal nerve activity (“VNA”) at a level approximately 2 cm above the aorta. In a Group 1 dog with D70CCTP implantation, a blood pressure sensor lead was inserted into the subclavian artery for blood pressure recordings but the results were not used in this study.

### VNS Protocol

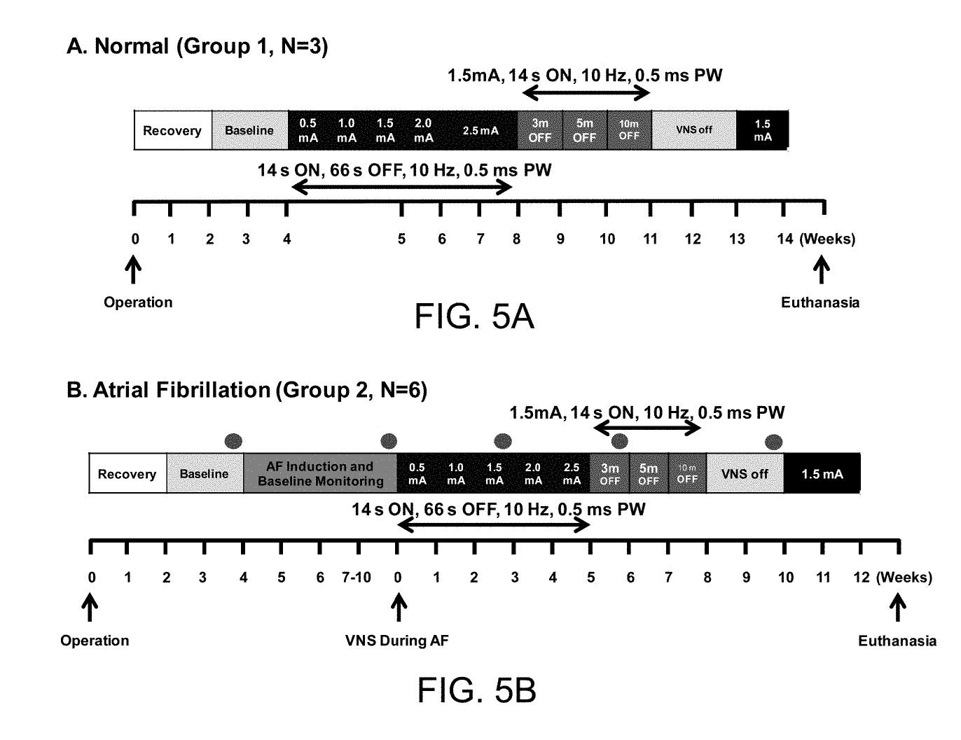

FIGS. 5A and 5B show the protocols of the study. Specifically, FIG. 5A shows an intermittent VNS (14 s ON-time, 66 s OFF-time, 10 Hz, 0.5 ms pulse width) for 3 normal ambulatory dogs (Group 1). The output current (mA) was progressively increased from week 4 to 8 until 2.5 mA. The output was then reduced to 1.5 mA and the OFF-time was progressively increased to 3 min, 6 min and 10 min. The VNS was stopped for week 13 to evaluate the effects of VNS withdrawal. The stimulation was reinitiated in week 14 for one week before euthanasia.

Shown in FIG. 5B, a VNS protocol was performed for 6 dogs with pacing-induced sustained AF (Group 2). As shown, a high rate (600 bpm, 2× the diastolic threshold output) atrial pacing was performed using a modified Secura implantable cardioverter defibrillator (Medtronic Inc, Minneapolis, Minn.) starting in week 4. The pacing protocol was halted in 2 weeks to determine if persistent (>48 hrs) AF was induced. To maintain a consistent protocol, the pacing was always terminated on Wednesdays and checks were performed for persistent AF on Thursday and Friday. If persistent AF was induced, data was continuously recorded data during the subsequent two days (Saturday and Sunday) to determine a first baseline AF reading.

The above procedure was repeated once to obtain second baseline AF recordings. VNS protocol was then started roughly 7-10 weeks after the first surgery. The programmed parameters of the VNS are shown in FIGS. 5A and 5B. In each week, the high rate atrial pacing was turned off on Wednesdays, with echocardiogram and blood samples being taken Thursdays. DSI monitoring was also on over the weekend. The VNS output was adjusted on Monday and the rapid atrial pacing was reinitiated from Monday to Wednesday to ensure that AF persisted. The same protocol then repeated itself according to that illustrated in FIG. 5B. At the end of protocol, stellate ganglia samples were harvested for histological analysis.

### Data Analysis

Recordings were analyzed from all channels using custom-written software, which selected the R waves and calculated the RR intervals automatically. All selections were then confirmed by manual examination. Nerve activities were considered present if there was a 3-fold increase in the amplitude over baseline noise. Both the frequency of sympathetic discharge episodes and the corresponding heart rate increments were analyzed after each discharge. To optimize nerve signals, data from SGNA and VNA were high-pass filtered at 150 Hz. The filtered signals were then rectified, integrated with a 100-ms time constant, and summed to represent integrated nerve activity of 10-s segments. For dogs in Group 1, integrated nerve activities and ventricular rate were determined during VNS OFF-time over a 24-hr period. For dogs in Group 2, integrated nerve activities and ventricular rate were determined for 2 min at the beginning, 20-min past and 40-min past the hour from each hour between 6 AM and 1 PM. RR-intervals were averaged over 1 minute windows for a 24 hour period to construct the RR-interval distribution curves.

### Echocardiography

Echocardiogram was used to evaluate left ventricular functions for dogs in Group 2. Conventional 2D and M-mode were performed using ACUSON Cypress echocardiography system (Siemens Medical Solutions USA, Inc., Mountain View, Calif.) and transducer (7V3c ACUSON, Siemens Medical Solutions USA, Inc., Mountain View, Calif.). Systolic and diastolic parameters including left ventricular (“LV”) end systolic diameter (“LVESd”), LV end diastolic diameter (“LVEDd”), interventricular septum thickness (“IVS”), LV Posterior Wall (“LVPW”), ejection fraction (“EF”), fractional shortening (“FS”) were obtained from M-mode using parasternal long axis view at least 10 consecutive cardiac cycles.

### Immunohistochemistry

Left stellate ganglion samples were harvested and fixed in 4% formalin for 45 min, followed by storage in 70% alcohol. The tissues were processed routinely, paraffin embedded and cut into 5 μm thick sections. Immunohistochemical staining was performed with antibodies against SK2 using rabbit polyclonal anti-KCNN2 (Abcam, Cambridge, Mass.) and antibodies against tyrosine hydroxylase (“TH”) using mouse monoclonal anti-TH (Accurate Chemical, Westbury, N.Y.).

A characteristic histological change of left cervical VNS was considered a significant increase of the percentage of TH-negative ganglion cells within the left stellate ganglion. To confirm these findings, 5 high-power (20×) fields were randomly selected with the highest ganglion cell density in the LSG from each dog. All cells were manually counted with any part of the cells visible in the picture. The percentage of TH-negative cell in that slide was then determined. The mean of those 5 selected fields was used as the value for that LSG.

### Statistical Analysis

Data were expressed as mean ±standard deviation. Statistical comparison of variables during baseline, atrial fibrillation and VNS was analyzed using paired t test. Analyses of variance with Bonferroni post hoc test were used to compare the results of immunostaining of the LSG. Paired t-test for pairwise comparisons were performed to compare the RR-intervals between different stages of experiments. Correlation coefficients between percent TUNEL-positive non-ganglion cells and ganglion cells were calculated accounting for the correlation of data from the same dog. A bootstrap method was used calculate the confidence interval (“CI”) of the correlation coefficient. The statistics were computed using the PASW Statistics (version 18; SPSS Inc, Chicago, Ill.) and SAS 9.2 (SAS Inc, Cary, N.C.). A two-sided P≦0.05 was considered as statistically significant.

### Results

### Effects of VNS on SGNA in Group 1

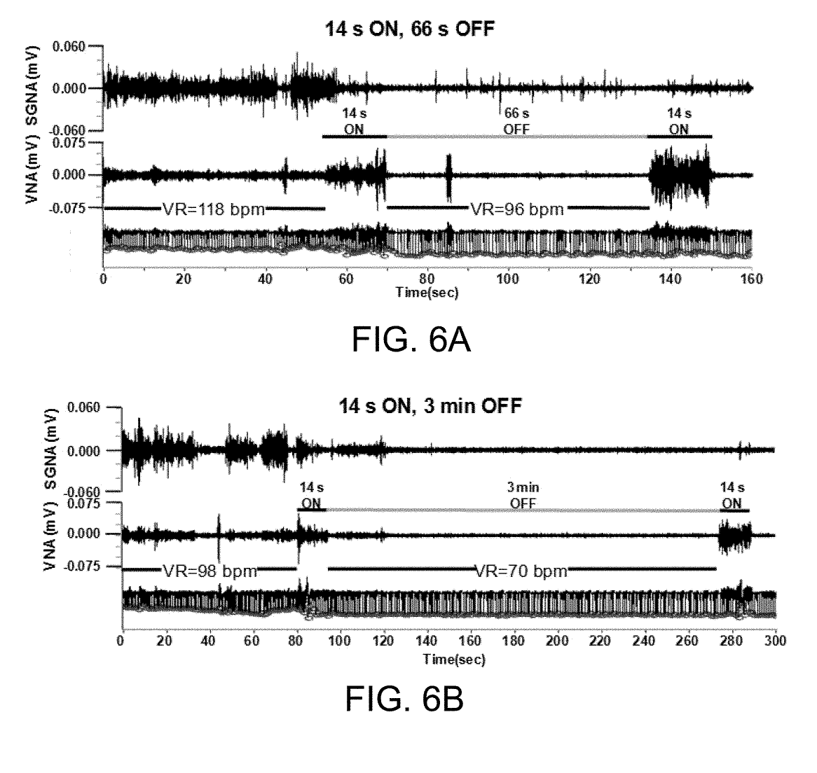

Dogs in Group 1 were used to determine the optimal programming parameters that most effectively suppressed SGNA during VNS OFF time. During baseline, iSGNA, and ventricular rate were 1.0±0.1 mV-s and 91±9 bpm, respectively. It was found that stimulated vagal nerve with 1.5 mA 14 s ON-time, 66 s OFF-time (FIG. 6A) and 3 min OFF-time (FIG. 6B) provided the most effective results of SGNA and ventricular rate reduction during OFF time period in this study. The burst of sympathetic discharges were demonstrated by SGNA firing concomitant with high ventricular rate. After 14 s of VNS, firing of SGNA as well ventricular rate was suppressed during the OFF-time. This SGNA suppression was concomitant with ventricular rate reduction. iSGNA and ventricular rate was reduced to 0.9±0.1 mV-s and 88±16 bpm in VNS 66 s OFF-time, and 0.8±0.1 mV-s and 83±13 bpm in VNS 3 m OFF-time. The iVNA at baseline, during VNS 66 s OFF-time and during VNS 3 m OFF-time were not significantly different (0.7±0.2 mV-s, 0.7±0.3 mV-s, and 0.7±0.1 mV-s, respectively).

Effects of VNS on SGNA and ventricular rate During AF (Group 2)

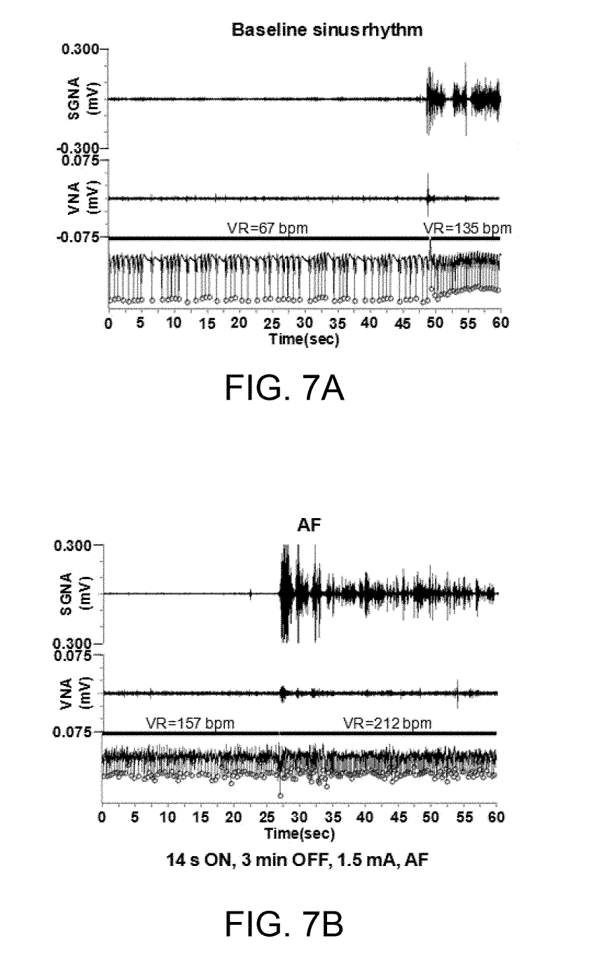

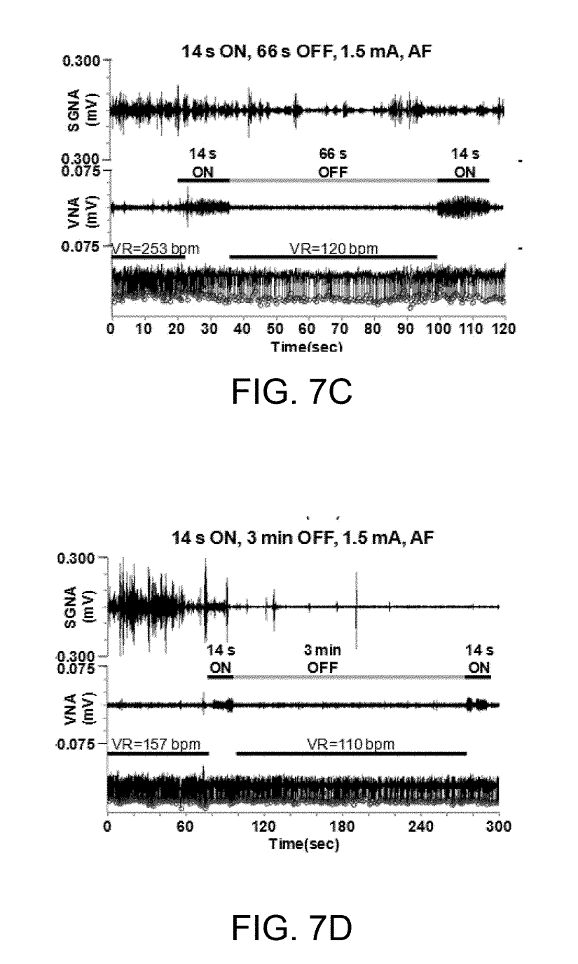

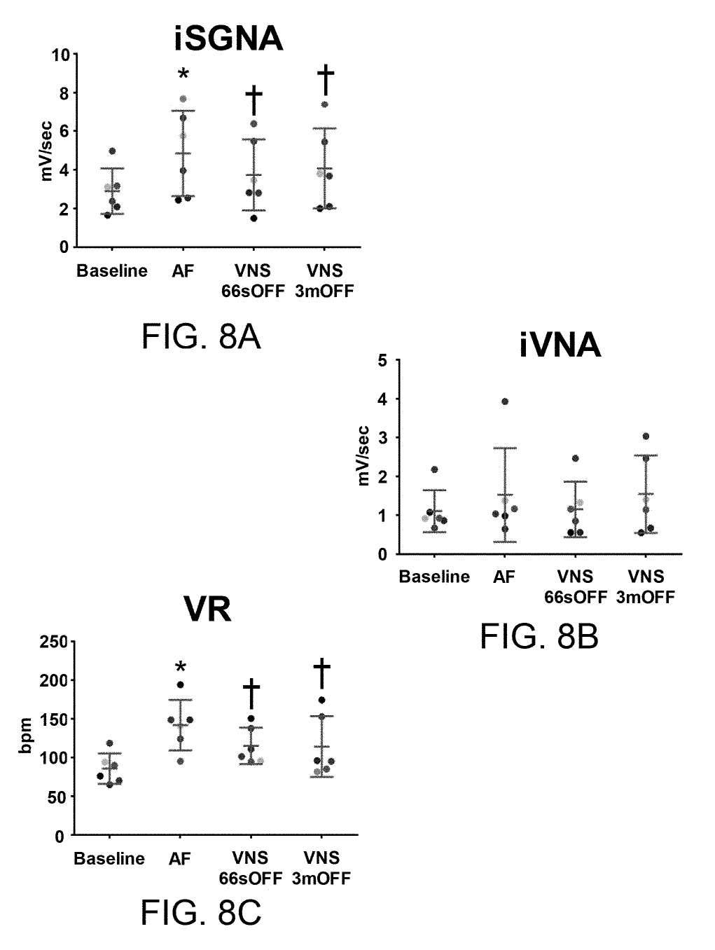

FIG. 7A illustrates nerve activities and ventricular rate of a Group 2 dog during baseline sinus rhythm. There were short periods of co-firing of SGNA and VNA resulting ventricular rate acceleration. Ventricular rate acceleration was also noted during persistent AF (FIG. 7B). iSGNA was significantly increased from 2.9±1.2 mV-s during baseline to 4.8±2.2 mV-s during AF (p=0.023) (FIG. 8A). iVNA was not different during baseline, AF, and after VNS (FIG. 8B). Ventricular rate increased from 86±20 bpm during baseline sinus rhythm to 142±33 bpm during AF (p=0.029) (FIG. 8C). VNS in both 66 s OFF-time and 3 m OFF-time suppressed sympathetic nerve activity and lowered ventricular rate during AF (FIGS. 7C and 7D). As compared to periods of AF, VNS reduced iSGNA and ventricular rate to 3.7±1.8 mV-s (p=0.021) and 115±24 bmp (p=0.016) in VNS 66 s OFF-time, and to 4.1±2.0 mV-s (p=0.037) and 114±39 bpm in 3 m OFF-time (p=0.039) (FIGS. 8A and 8C). When compared with AF, the VR during VNS withdrawal were significantly reduced while iSGNA were comparable. These results are summarized in Table. 1

TABLE 1
Nerve activity and Ventricular Rate

| | | | VNS | VNS | VNS |
|---|---|---|---|---|---|
| Parameters | Baseline | AF | 66-s OFF | 3-mins OFF | withdrawal |
| iSGNA (mV-s) | 2.89 ± 1.17 | 4.84 ± 2.19* | 3.74 ± 1.83† | 4.07 ± 2.06† | 4.09 ± 1.73 |
| iVNA (mV-s) | 1.10 ± 0.54 | 1.52 ± 1.21 | 1.15 ± 0.72 | 1.54 ± 1.00 | 1.58 ± 1.17 |
| VR (bpm) | 86 ± 20 | 142 ± 33* | 115 ± 24† | 114 ± 39† | 115 ± 23† |

### RR Interval Distributions

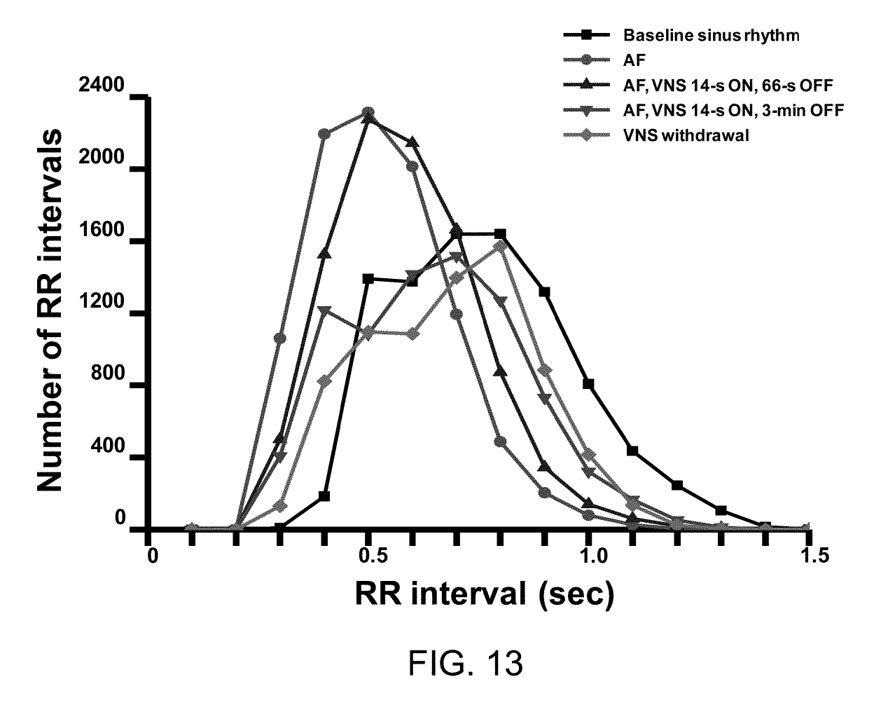

All RR-intervals were analyzed over a 24-hr period and plotted the average distribution all 6 dogs studied (FIG. 13). The RR-intervals were 0.77 sec [CI, 0.60 to 0.93] during baseline sinus rhythm, 0.46 sec [CI, 0.34 to 0.58] during AF, 0.53 sec [CI, 0.40 to 0.64] during VNS 14-s ON/66-s OFF, 0.59 sec [CI, 0.42 to 0.76] during VNS 14-s ON/3-min OFF and 0.64 sec [CI, 0.50 to 0.77] during VNS withdrawal. As shown in FIG. 13, compared to baseline (black line, square), the curve shifted to the left and the base was narrowed during sustained AF (red line, circle). VNS 14-s ON/66-s OFF (blue line, right-side up triangle), VNS 14-s ON/3-min OFF (purple line, upside-down triangle) and VNS withdrawal (pink line, diamond) gradually moved the curves back towards the baseline sinus rhythm (black line, square). Significant differences were found between red and black curves (−0.30 s, [CI−0.53 to −0.08, p=0.0178]) and between blue and black curves (−0.24, [CI−0.44 to -0.05, p=0.0252]). However, there were no significant differences of RR-intervals between purple and black curves (−0.17 s, [CI−0.41−0.06, p=0.1176]) or between pink and black curves (−0.13, [CI−0.33 to 0.08, p=0.1673]). These data indicate that VNS progressively lengthened the RR-intervals during persistent AF. These effects were not reversible by 2 weeks of VNS withdrawal.

### Effect of VNS on Left Ventricular Functions

Table 2 details left ventricular parameters measured in this study. AF dogs demonstrated both systolic dysfunction and left ventricular dilatation as indicated by reduction of EF and FS, and increased in LVESD, LVEDD. In spite of prolonged AF, there was no further reduction of LVEF during VNS.

TABLE 2
Echocardiographic parameters

| | | | AF_VNS_{—} | AF_VNS_{—} |
|---|---|---|---|---|
| Parameters | Baseline | AF | 66sOFF | 3mOFF |
| LVEDD | 4.3 ± 0.3 | 4.7 ± 0.3* | 4.6 ± 0.8 | 4.9 ± 0.8 |
| LVPWd | 0.7 ± 0.1 | 0.7 ± 0.1 | 0.6 ± 0.1 | 0.7 ± 0.2 |
| IVSd | 0.7 ± 0.2 | 0.7 ± 0.1 | 0.7 ± 0.1 | 0.7 ± 0.1 |
| LVESD | 2.9 ± 0.2 | 3.5 ± 0.3* | 3.5 ± 1.1 | 3.6 ± 0.9 |
| LVPWs | 1.0 ± 0.1 | 1.0 ± 0.1 | 0.9 ± 0.1 | 1.0 ± 0.2 |
| IVSs | 1.1 ± 0.1 | 1.0 ± 0.1 | 1.0 ± 0.1 | 1.1 ± 0.2 |
| % EF | 60 ± 5 | 51 ± 6* | 50 ± 18 | 51 ± 11 |
| % FS | 32 ± 4 | 26 ± 4* | 26 ± 11 | 27 ± 6* |

LVEDD, left ventricular end diastolic diameter; LVPWd, left ventricular posterior wall dimension; IVSd, interventricular septum during diastole; LVESD, left ventricular end systolic diameter; IVSs, interventricular septum during systole; EF, ejection fraction; FS, fractional shortening.
*p < 0.05 vs. baseline.

### Prolonged Pauses During AF

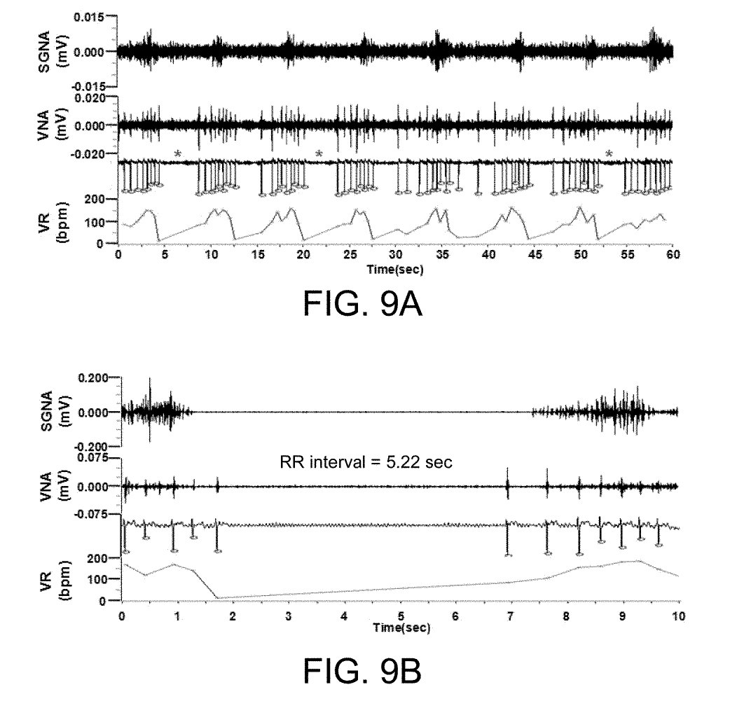

Prolonged (greater than 3s) pauses (asterisks, FIG. 9A) were observed with increased frequency during VNS as compared with baseline. These prolonged pauses were found 89±79 times/24 hours and 213±245 times/24 hours during VNS 66 s-OFF and VNS 3 m-OFF, respectively. In comparison, prolonged pause were found only 2±2 time/24 hours during baseline sinus rhythm and 16±14 times/24 hours during AF. The iSGNA was lower during the pause (0.8±0.2 mV-s) than not during the pause (7.4±2.9 mV-s, p=0.06). In addition, there was a significant circadian distribution of the long pauses with most of the episodes occurring in the early morning. FIG. 9B shows roughly a 5 s pause, which was the longest pause found in this study.

### Histology

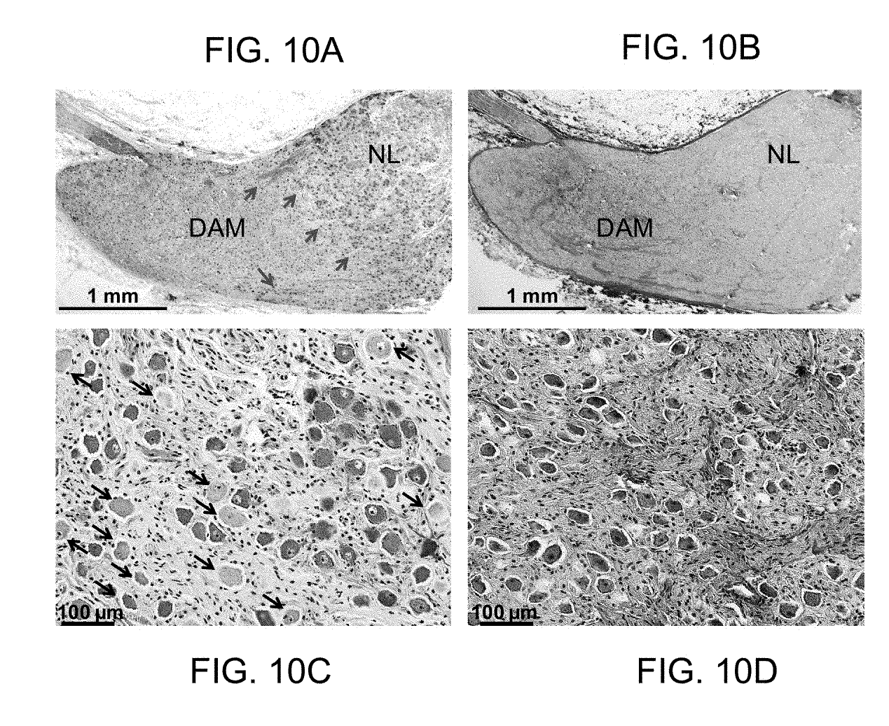

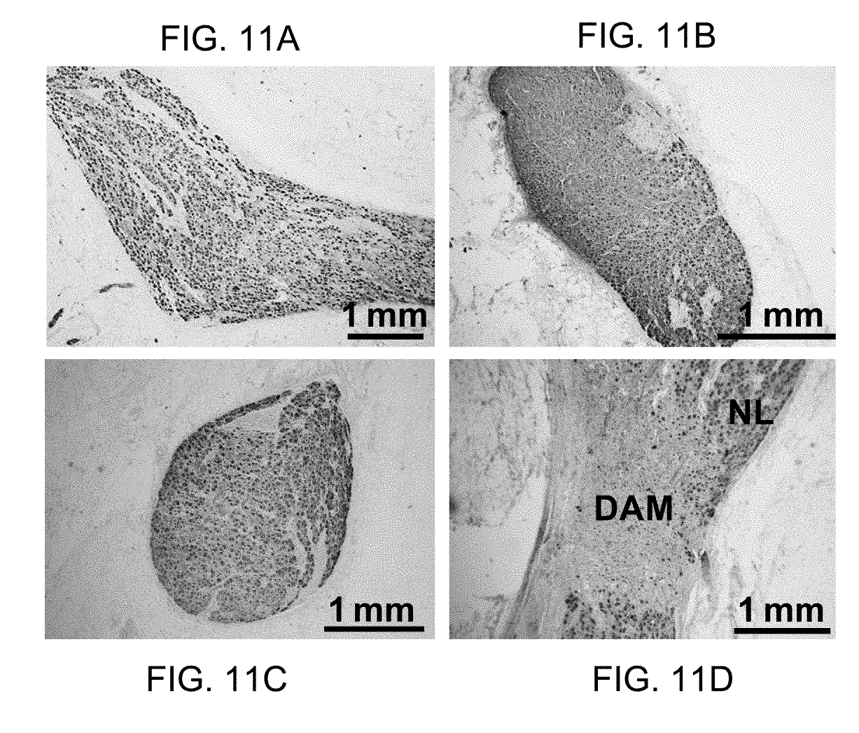

Tyrosine hydroxilase (“TH”) immunostaining cells of LSG was evaluated for Group 1, Group 2 and normal control dogs. FIGS. 10A and 10B show TH immunostaining and trichrome staining, respectively, of the LSG at low (40×) magnification. The red arrows mark the boundary between the damaged region (“DAM”) and normal region (“NL”). FIGS. 10C and 10D show a high (200×) magnification view of the same slide in the fibrotic regions. Note that there is a large number of TH (−) cells (black arrows) in FIG. 10C. Most of the ganglion cells appear pyknotic and contain reduced TH. FIG. 10D shows increased fibrosis (blue) in the fibrotic region. The size of DAM and NL regions were measured in FIG. 10A, and determined that 50.38% of the LSG was in the DAM region. One LSG from Group 1 and five LSG from Group 2 were available for analysis. For all 6 LSG studied, the DAM region accounted for 32.88±14.59% of the LSG. In comparison, 0% of the RSG of the same dogs are in the DAM region (p=0.004).

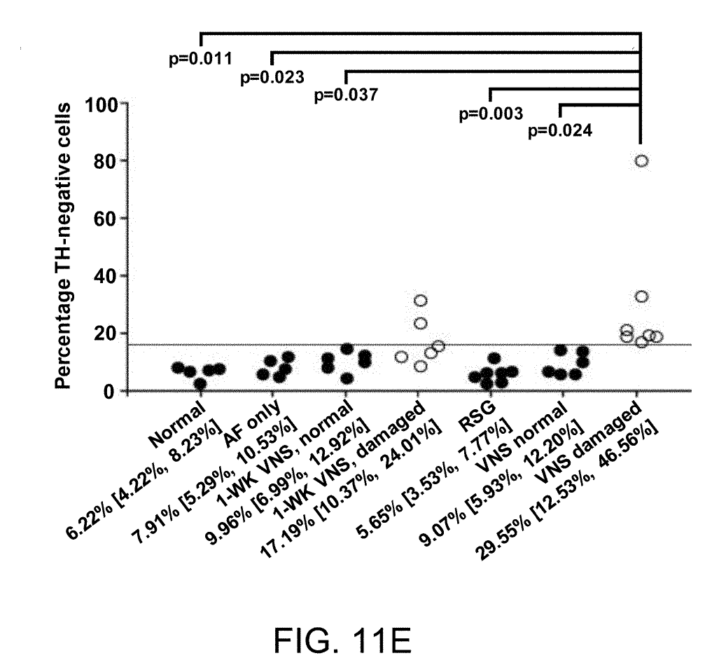

FIG. 11A shows a LSG sample from a normal dog. FIG. 11B shows a LSG sample from a dog with AF and LSG recordings, but without VNS. In spite of direct LSG recordings, the latter group of dogs did not have large confluent stellate ganglion damage. No large and confluent damages were found in RSG of the present study (FIG. 11C) as well. FIG. 11D shows the LSG from a Group 2 dog, showing a large DAM region. The percent TH-negative ganglion cells in all groups are shown in FIG. 11E. Only LSG in 2 VNS groups had percent of TH-negative cells of greater than 16%. Statistically significant differences of the percentage of TH-negative ganglion cells were found between the damaged region of the LSG in the present study and non-VNS groups.

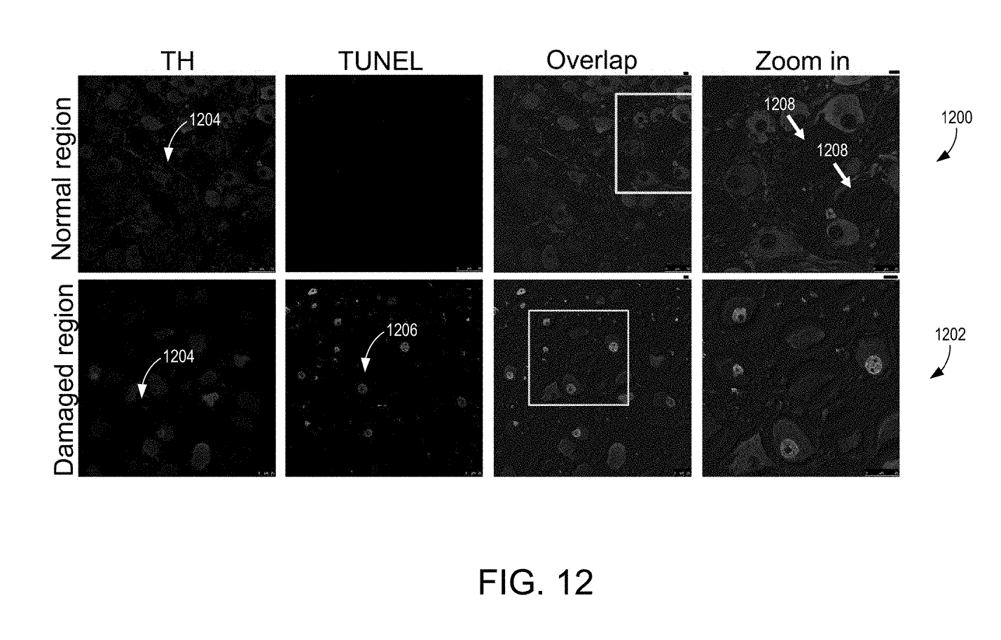

The slides from the same LSG shown in FIG. 10A were then double stained for TH and terminal deoxynucleotidyl transferase dUTP nick end labeling (“TUNEL”). As shown in confocal immunofluorescent images of FIG. 12, the ganglion cells in normal region 1200 mostly stained positive for TH (Red, labeled 1204) and none stained for TUNEL (Green, labeled 1206). TH-negative ganglion cells (labeled 1208) were also TUNEL-negative. In contrast, cells in the damaged regions 1202 stained negative for TH, confirming the results of FIG. 10, but positive for TUNEL. It is noteworthy that multiple ganglion cells and small endothelial or Schwann cells in the damaged region stained positive for TUNEL. All 7 VNS dogs had TUNEL-positive ganglion cells, but no TUNEL-positive ganglion cells were found in 5 normal control LSG. TUNEL-positive ganglion and non-ganglion cells were measured on all 12 LSGs using images taken with high power (20×) objectives. There were 5 images analyzed per dog except for 1 normal dog that had 4 images due to small sample size. In VNS group (N=7), 19.50% [CI 9.78 to 29.22] of ganglion cells and 16.18% [CI 4.02 to 28.35] of non-ganglion cells were TUNEL-positive. The correlation coefficient between % TUNEL-positive ganglion cells and non-ganglion cells was 0.48 [CI, 0.16 to 0.64]. In contrast, there were no TUNEL-positive ganglion cells and rare TUNEL-positive non-ganglion cells (0.39%, [CI 0.10 to 0.68]) in the normal LSG (N=5).

TUNEL staining was performed for 8 RSG samples. In spite of the absence of apparent RSG damage by TH staining, rare TUNEL-positive cells were found in 4 of the 8 samples. The number of TUNEL-positive cells was low, averaging 1±3 cells (range 0 to 13 cells) per image. The TUNEL-positive cells accounted for 2.16±0.05% (range 0-17%) of the ganglion cells. Percent TUNEL-positive cells in RSG was significantly less than that in the LSG (p=0.0007).

### Discussion

An important finding presented herein is that intermittent open-loop VNS with a short ON-time and long OFF-time results in stellate ganglion remodeling, leading to reduced SGNA and ventricular rate during sustained atrial fibrillation in ambulatory dogs. Specifically, application of VNS with short (for example, 14 s) ON-time and long (for example, 66-s or 3-min) OFF-time can significantly reduce ventricular rate during sustained AF.

Previous studies showed that continuous VNS for one week can result in significant LSG changes that include upregulation of the small conductance calcium activated K (“SK”) channels and reduced iSGNA, leading to reduced episodes of paroxysmal AF. Results described here show that prolonged open-loop intermittent VNS causes large area of increased fibrosis, reduced TH immunoreactivity and increased TH-negative cells in the LSG. These latter findings are relevant to clinical practice, as the patients stimulated chronically with intermittent VNS with short ON-time and long OFF-time permit the ability to remodel LSG structure and function, with important in the clinical consequences for controlling AF.

Specifically, a rapid ventricular rate is a common complication of AF. While many patients can be controlled by atrioventricular (“AV”) nodal blocking agents, some patients are refractory to drug therapy. Extremely symptomatic patients may require AV node ablation followed by pacemaker implantation. The downside of the latter procedure is that the patients become pacemaker-dependent and have a high incidence of sudden death during follow up. In addition, progressive ventricular dysfunction is also commonly observed in these patients after prolonged right ventricular apex pacing. Therefore, an approach using an intermittent open-loop VNS, as described in the present disclosure, may serve as an alternative approach to AV node ablation in such patients.

In the present study, predominantly LSG samples, rather than RSG samples, from VNS-treated dogs showed large and confluent regions of damage. TUNEL staining was positive for both SG samples, but the percentage of TUNEL-positive ganglion cells were much higher in the LSG than the RSG. It is unlikely that reduced TH staining in the LSG was due to fixation or staining artifacts because normally stained regions were also present in each LSG. It is possible that these changes were secondary to either sustained AF or prolonged irritation caused by recording electrodes. Therefore, LSG samples from a previous study, where dogs were subjected same types of recordings and prolonged rapid atrial pacing to induce sustained AF, were retrospectively stained. No damage was observed in those samples. LSG samples from a study in which a short duration (5 days) of continuous left cervical VNS without making recordings of the LSG, were also retrospectively stained. Because no thoracotomy was performed in the latter study, any changes in the LSG could not be due to local fibrosis or inflammation caused by prolonged contact with the recording electrodes. As damaged regions were also found in the LSG of the latter study, these findings further support the conclusion that VNS can cause LSG damage.

Vagal nerves have significant sympathetic components. In dogs, these sympathetic nerve fibers were distributed mostly in the periphery of the vagal nerve, close to the VNS electrodes. Because of the direct connection between LSG and vagal nerves, stimulation of the sympathetic component in the vagal nerve may retrogradely activate the ganglion cells in the LSG at high rates, followed by abrupt termination of SGNA. However, chronic intermittent high rate excitation may cause neuronal cell death (excitotoxicity) due to intracellular calcium accumulation. Excitotoxicity can also be demonstrated in the in vitro preparation, where 12-14 hours of electrical stimulation can cause cell death accompanied by increased percentage of TUNEL-positive cells. LSG samples changes in the present study, including the pyknotic and dense nuclei and positive TUNEL staining were consistent with excitotoxicity. There are some TUNEL-positive non-ganglion cells in normal LSG, consistent with physiological cell death. The percentage of TUNEL-positive cells in the LSG of VNS dogs greatly exceeded that in the LSG of normal dogs, suggesting that most of the TUNEL-positivity in the VNS group was not only due to physiological cell death. While excitotoxicity is a possible mechanism of SG damage caused by VNS, glutamate or other neurotransmitters in the excised ganglion was not measured. Therefore, whether or not excitotoxicity underlies the mechanism of cell death in the SG could be determined by future studies.

It was found in the present study that VNS reduced the average VR in dogs with persistent AF. Profound bradycardia and AV block have also been reported in patients with refractory epilepsy receiving VNS therapy. While VNS did not completely normalize the LVEF, the mean LVEF was maintained at 50% or higher throughout the study. In comparison, dogs with 3 months of AF and intact atrioventricular node were expected to have much lower LVEF (around 30%). These data suggest that VNS might have prevented the progression of tachycardiomyopathy. However, it was also found that there was an increased incidence of prolonged pauses during VNS. Because the SGNA during the pause was lower than that without pause, the prolonged RR-interval might have occurred as a consequence of SGNA suppression. If VNS was used in patients with chronic AF, symptomatic bradycardia could be considered as one of the anticipated side effects of VNS.

VNS is known to reduce T wave alternans and improve sympathovagal balance in patients with drug-refractory partial-onset seizures. Chronic VNS can prevent ventricular fibrillation and sudden cardiac death in conscious dogs with a healed myocardial infarction. A recent clinical trial with both right and left cervical VNS showed that VNS may be beneficial in heart failure (“HF”) control, but another trial showed right cervical VNS alone failed to improve systolic function in patients with HF. The LSG is an important source of cardiac sympathetic innervation. The left SGNA is a direct trigger of cardiac arrhythmias in ambulatory canine models. Left cardiac sympathetic denervation (“LCSD”) that includes partial resection of the LSG could be beneficial in managing patients with long QT syndrome and catecholaminergic polymorphic ventricular tachycardia. LCSD or bilateral sympathectomy has also been used in patients with organic heart diseases to improve mortality and to control refractory ventricular arrhythmias. These studies indicated that LSG is an important arrhythmogenic structure and that LSG ablation may be effective in arrhythmia control. Results from the present study showed that intermittent left VNS can remodel the LSG and reduce sympathetic outflow. These effects may be useful in managing cardiac arrhythmia.

Features suitable for such combinations and sub-combinations would be readily apparent to persons skilled in the art upon review of the present application as a whole. The subject matter described herein and in the recited claims intends to cover and embrace all suitable changes in technology.

1-20. (canceled)

21. A system for controlling a ventricular rate during atrial fibrillation, the system comprising:
one or more electrodes configured to be positioned at locations associated with a vagal nerve of the subject;
an electrical source coupled to the one or more electrodes and configured to generate electrical signals for electrically stimulating the vagal nerve; and
a processor configured to:
select a treatment protocol based on a determined condition of the subject, wherein the treatment protocol comprises intermittent periods of electrical stimulation separated by periods of non-stimulation;
direct the electrical source to apply an electrical stimulation, according to the selected the treatment protocol, using the one or more electrodes.

22. The system of claim 1, wherein the treatment protocol selected by the processor is further configured to control a sympathetic nerve activity in the subject.

23. The system of claim 1, wherein the electrical stimulation is described by parameters comprising one or more of a timing, an intensity, a duration, a frequency, or a waveform, or any combinations thereof.

24. The system of claim 1, wherein the treatment protocol selected by the processor is further configured to remodel at least one neural structure.

25. The system of claim 4, wherein the at least one neural structure includes a stellate ganglion.

26. The system of claim 1, wherein the treatment protocol selected by the processor is further configured to reduce a neural activity in the subject.

27. The system of claim 6, wherein the neural activity includes a stellate ganglion activity.

28. The system of claim 1, wherein the processor is further configured to reduce the ventricular rate using the electrical stimulation using the selected treatment protocol.

29. The system of claim 1, wherein time durations of the intermittent periods of electrical stimulations are in a range between 1 and 20 seconds.

30. The system of claim 1, wherein time durations of the periods of non-stimulation are in a range between 60 seconds to 15 minutes.

31. A method for controlling a ventricular rate during atrial fibrillation, the method comprising:
selecting a treatment protocol based on a determined condition of a subject, the treatment protocol comprising intermittent periods of electrical stimulation separated by periods of non-stimulation and arranged asynchronously; and
applying an electrical stimulation, according to the selected the treatment protocol, using an electrical source and at least one electrode positioned at locations associated with a vagal nerve of the subject to control a ventricular rate during atrial fibrillation.

32. The method of claim 11, wherein the treatment protocol is further configured to control a sympathetic nerve activity.

33. The method of claim 11, wherein the electrical stimulation is described by parameters comprising one or more of a timing, an intensity, a duration, a frequency, or a waveform, or any combinations thereof.

34. The method of claim 11, wherein the treatment protocol is further configured to remodel at least one neural structure.

35. The method of claim 14, wherein the at least one neural structure includes a stellate ganglion.

36. The method of claim 11, wherein the treatment protocol is further configured to reduce a neural activity.

37. The method of claim 16, wherein the neural activity includes a stellate ganglion activity.

38. The method of claim 11, wherein the method further comprises reducing the ventricular rate using the electrical stimulation.

39. The method of claim 11, wherein time durations of the intermittent periods of electrical stimulations are in a range between 1 and 20 seconds.

40. The method of claim 1, wherein time durations of the periods of non-stimulation are in a range between 60 seconds to 15 minutes.
